(19) United States
(12) Patent Application Publication
(10) Pub. No.: US 2026/0099918 A1
(43) Pub. Date: Apr. 9, 2026

(54) WEARABLE DEVICE USING VISION-BASED TACTILE SENSORS TO DETECT OBJECT FIRMNESS

(71) Applicant: Khalifa University of Science and Technology, Abu Dhabi (AE)

(72) Inventors: Mashood Mohammad Mohsan, Abu Dhabi (AE); Basma Bashir Mohamed Hasanen, Abu Dhabi (AE); Irfan Hussain, Abu Dhabi (AE); Naoufel Werghi, Abu Dhabi (AE); Lakmal Seneviratne, Abu Dhabi (AE); Taimur Hassan, Abu Dhabi (AE)

(21) Appl. No.: 18/908,435

(22) Filed: Oct. 7, 2024

(51) Int. Cl. G06T 7/00; G06T 7/246; G06T 7/579; G06V 10/764; G06V 10/77; G06V 20/68

(52) U.S. Cl. CPC G06T 7/0012; G06T 7/246; G06T 7/579; G06V 10/764; G06V 10/7715; G06V 20/68; G06T 2207/30004; G06T 2207/30188

(57) ABSTRACT

Techniques are described herein for a method may include generating, by using a vision-based tactile sensor (VBTS) of a wearable device, palpation data corresponding to a deformation detected by the VBTS upon a contact of the VBTS with an object that includes at least one of: a fruit, a legume, or a vegetable, generating an input to a machine learning (ML) model based on the palpation data; determining an output of the ML model in response to the input such that the output may indicate a firmness of the object. In some embodiments, the method may include presenting, by using a user interface (UI) of the wearable device, an indication of the firmness.

The present disclosure is directed to wearable sensors, components, devices, and methods. More particularly, the present disclosure describes a wearable device using vision-based tactile sensors to detect object firmness.

### BACKGROUND

Accurately assessing fruit ripeness is a relevant task in the agricultural industry, as it significantly impacts fruit quality, shelf life, and consumer satisfaction. Fruits harvested at the optimal stage of ripeness are more likely to withstand transportation and storage without damage, thereby preserving their market value and reducing post-harvest losses. For fruits like tomatoes, mangoes, bananas, and apples, dynamic skin color changes during ripening can be detected using several computer vision (CV) based solutions. However, these CV-based solutions are ineffective for fruits whose ripeness is not visually apparent, such as avocados and kiwis. Mechanical, acoustic, vibrational share similar deficiencies.

### BRIEF SUMMARY

In some embodiments, a method may include generating, by using a vision-based tactile sensor (VBTS) of a wearable device, palpation data corresponding to a deformation detected by the VBTS upon a contact of the VBTS with an object that includes at least one of: a fruit, a legume, or a vegetable, generating an input to a machine learning (ML) model based on the palpation data; determining an output of the ML model in response to the input such that the output may indicate a firmness of the object. In some embodiments, the method may include presenting, by using a user interface (UI) of the wearable device, an indication of the firmness.

In some embodiments, the input may include the palpation data. In some examples, the method may further include displaying, by the UI, a characterization of the firmness of the fruit, legume, or vegetable.

In some embodiments, the palpation data is generated based on the VBTS being in contact with the object without the object being removed from a parent plant to which object is attached and without a damage to the object.

In some embodiments, the ML model may be executed on a system remote from the wearable device, and the method may further include sending the palpation data as the input to the system and receiving the output of the ML model from the system.

In some embodiments, the method may further include executing, locally on the wearable device, the ML model.

In some embodiments, a time interval between generating the palpation data and presenting the indication may be in a range between 0.1 and 20.0 seconds In some embodiments, a device may include a first wearable interface, a vision-based tactile sensor (VBTS) attached to the first wearable interface and configured to generate palpation data upon contact of the VBTS with an object that includes at least one of a fruit, legume, or vegetable, a second wearable interface, and a user interface (UI) attached to the second wearable interface and configured to present an indication of a firmness of the object, the indication generated by a machine learning model (ML) based on the palpation data.

In some embodiments, processing circuitry may be attached to the second wearable interface and configured to send the palpation data to a system executing the ML model such that the processing circuitry may be configured to receive the firmness as an output of the ML model and cause the presentation of the firmness at the UI.

In some embodiments, the device may further include processing circuitry attached to the second wearable interface and configured to execute the ML model.

In some embodiments, the first wearable interface may include a housing configured to receive a thumb at a first end such that the housing is configured to receive the VBTS at a second end, and at least one camera.

In some embodiments, the second wearable interface may include a housing configured to couple to an appendage, a screen configured to display a graphical user interface (GUI) and at least partially disposed within the second wearable interface such that the UI may include the GUI, and wherein the second wearable interface is configured to at least partially house processing circuitry and a transceiver.

In some embodiments, the first wearable interface and the second wearable interface may be linked together by a coupler selected from a group comprising: a wired connection, a wireless connection, a fabric, a tether, or combinations thereof.

In some embodiments, the VBTS may include a surface configured to contact the object such that the surface is an elastomer configured to deform when placed in contact with the object.

In some embodiments, a non-transitory computer readable medium having stored thereon computer-readable instructions that, when executed by a processor, cause the processor to perform operations including generating, by using a vision-based tactile sensor (VBTS), palpation data corresponding to a deformation detected by the VBTS upon a contact of the VBTS with an object, generating an input to a machine learning (ML) model based on the palpation data, determining an output of the ML model in response to the input, wherein the output indicates a firmness of the object, and causing a presentation of an indication of the firmness at a user interface (UI).

In some embodiments, the object may be a fruit or vegetable. The operations may further include determining a harvest time of the object based on the firmness such that the harvest time is an estimated time predicted to elapse before the object is ripe, and presenting, using the UI, the harvest time.

In some embodiments, the operations may further include storing the harvest time in a memory, and in response to comparing the harvest time to a time threshold, transmitting a notification to a client device.

In some embodiments, the palpation data includes one or more tactile images. The operations may further include receiving, from the VBTS, a first tactile image when VBTS initially contacts the object, receiving, from the VBTS, additional tactile images while VBTS contacts the object during a time interval. In some examples, the one or more tactile images includes the first tactile image and the additional tactile images and causing a token operation to be performed by the ML model on the one or more tactile images to generate firmness data associated with the firmness of the object.

In some embodiments, the operations may further include identifying, based at least partially on the one or more tactile images, a type of the object, in response to identifying the type, recalculating the firmness data, and presenting, using the UI, the type and the recalculated firmness data.

In some embodiments, the operations may further include causing training of the ML model using the one or more tactile images, and in response to the training, updating the firmness data.

In some embodiments, the operations may further include receiving, from the UI on a wearable device, a selection of a type of the object, in response to receiving the type of the object, recalculating the firmness, and presenting, using the UI, the recalculated firmness.

In some embodiments, various technical features, aspects, and advantages of the present disclosure are readily appreciated from the following detailed description. The present disclosure should not be considered limiting, and one or more embodiments discussed herein may be combined in various non-limiting ways. Some or all embodiments herein may be modified without departing from the scope of the present disclosure. The detailed description and drawings may be illustrative of the present disclosure such that advantages of the invention will be demonstrated.

### BRIEF DESCRIPTION OF THE DRAWINGS

The foregoing aspects and many of the advantages of the present disclosure will become more readily appreciated as these advantages become better understood by reference to the following detailed description, when taken in conjunction with the accompanying drawings.

FIG. 1 is a simplified example illustration of a wearable device, according to some embodiments.

FIG. 2 is a simplified example front view of a wearable device, according to some embodiments.

FIG. 3 is a simplified example side view of a wearable device, according to some embodiments.

FIG. 4 is a simplified example exploded view of a first wearable interface, according to some embodiments.

FIG. 5 is a simplified example illustration of a second wearable interface, according to some embodiments.

FIG. 6 is a simplified example exploded view of a portion of a second wearable device, according to some embodiments.

FIG. 7 is a simplified example set of wearable devices, according to some embodiments.

FIG. 8 is a simplified example group of tactile images used conjunction with a machine learning model, according to some embodiments.

FIG. 9 is a simplified example process for predicting firmness of an object, according to some embodiments.

FIG. 10 is a simplified example method for a wearable device, according to some embodiments.

FIG. 11 is a simplified example network interface for a wearable device, according to some embodiments.

In the drawings, like reference numerals refer to like parts throughout the various views and embodiments unless otherwise specified. Not all instances of an element are necessarily labeled to improve clarity in the drawings where appropriate. The drawings are not necessarily to scale, emphasis instead being placed upon illustrating the principles being described.

### DETAILED DESCRIPTION

Embodiments are described below in the context of wearable devices using vision-based tactile sensors to detect object firmness. Conventional firmness sensors such as mechanical devices may assess firmness through compression, puncture, and impact tests which may harm or even destroy the object that is being tested. Invasive mechanical devices, such as the Magness-Taylor (MT) penetrometer, measure rupture force by penetrating a probe into the fruit. These devices are operator-dependent, leading to variability results. Improvements like the force gauge on a controlled stand have been developed to enhance precision. Still, a significant drawback of these devices is their invasiveness, which may result in the disposal of the test samples after measurement. Noninvasive mechanical devices, such as durometers, measure parameters like resistance or bioyield force (e.g., specific force at which an initial cellular rupture occurs within a biological material) without significantly damaging the fruit. However, these devices rely on the operator's skill and technique, and they are usually designed for certain types of fruits, necessitating additional assembly for use with different varieties.

Other conventional firmness sensors such as acoustic and vibrational devices use sound waves and vibrations to non-destructively measure fruit firmness. Prior to determining firmness, vibrational and acoustic devices need to have a fruit's weight prior to measurement. Acoustic devices generate sound waves through impact excitation and capture the resulting acoustic signals to assess firmness leveraging the fruit's weight. Vibrational devices assess fruit using a vibration generator and measure the response using a detector and also including the fruit's weight. They rely on the resonance frequency of the fruit, which correlates with firmness. The acoustic and vibrational approach may be used as a non-invasive approach, however, it also can be affected by environmental factors such as temperature, humidity, and background noise, which may influence the accuracy of the measurements leading to product loss, cost losses, or similar.

Some conventional optical firmness sensors use visible (VIS) and near-infrared (NIR) spectra to non-destructively assess multiple quality attributes, including firmness. Reflectance-based devices measure diffuse reflectance spectra to build predictive models for firmness-related parameters. Examples include handheld NIR analyzers and portable VIS/NIR spectrometers. Transmittance-based devices measure light transmitted through the fruit, acquiring information on internal quality. Conventional optical firmness sensors for firmness estimation can be expensive and inconsistent due to variations in fruit surface color, texture, and shape. In addition, they may need specific calibration for different fruit types and batches, and environmental factors like dust, dirt, moisture, and surface damage can affect accuracy. Moreover, when ripeness cannot be determined by color, firmness becomes the primary indicator for assessing ripeness. Devices such as penetrometers may provide firmness readings but the devices typically use preparation of the surface of the fruit such as by removing layers of skin, penetrating into the fruit itself, or similar which may render the fruit inedible and damaged. These devices are also operator dependent and work optimally with a trained technician that knows the best practices to get the best readings. These conventional devices may not be used by individuals not trained in best practices and may use the devices differently which may lead to different ripeness readings which is not ideal.

To remedy the above limitations and deficiencies, embodiments of the present disclosure are directed towards a wearable device utilizing vision-based tactile sensors (VBTS) designed for quality inspectors, farmers, or customers to estimate fruit or vegetable firmness. The wearable device may be worn on an appendage of a user such that the user may handle a fruit to measure firmness. The tactile sensors on the wearable device may significantly enhance interaction and perception of fruit firmness regardless of location of the user. In addition, using VBTS, which may utilize red-green-blue (RGB) cameras to capture high-resolution palpation data, are particularly effective. As such, a non-destructive approach using VBTS for estimating fruit firmness and grading ripeness may be achieved by the user wearing the wearable device and mimicking human palpation.

The device, which may be worn on the thumb and a forearm, applies human-level pressure to the fruit surface, causing deformation of an elastomer near the thumb. The firmer a fruit is, the more deformation will occur on the elastomer. This deformation may be captured by the RGB camera, and a deep learning model processes the data to estimate firmness. The design of the thumb module ensures that the force applied to the fruit's surface mimics that of the thumb tip. The device features a versatile and body symmetrical design, allowing it to be comfortably worn on either the right or left hand. The modular structure ensures ergonomic flexibility, accommodating user's preferences. The wearable device ensures efficient and accurate evaluation of fruit firmness which is cost-effective for in-situ fruit grading. The wearable device includes electronics housed in a flexible white ABS material to ensure comfort. Additionally, the wearable device may include an adjustable strap mechanism that allows it to be wearable by anyone. The wearable device provides a significant improvement in determining fruit firmness in its non-destructive operation, compact and flexible profile, reliable AI-based firmness determination, and wireless and on-tree measurement capabilities. In addition, an optimal time to harvest may be determined which may maximize fruit quality and shelf life, ripeness during storage and transportation may be monitored which will reduce waste and enhance supply chain efficiency and enables consumers to check fruit ripeness in-situ at a point of purchase.

Moreover, the device can be valuable for vendors when purchasing fruits in bulk (e.g., containers or lot-wise), as the vendors may assess the ripeness of the entire lot by examining a few samples without destroying the product, which is the case for conventional devices. Consumers can also benefit by using the device to check if a fruit is ripe or overripe, which they currently perform by manually palpating the fruit at shops or stores, by using the device which is independent of the user operating the device.

According to embodiments of the disclosure herein, objects such as fruit, vegetables, or legumes are described as a particular use case for determining firmness on the object using a wearable device. However, the type of object should not be considered limiting. According to embodiments herein, the wearable device may similarly apply to measuring firmness of any other object including, for instance, meat or any innate or living object.

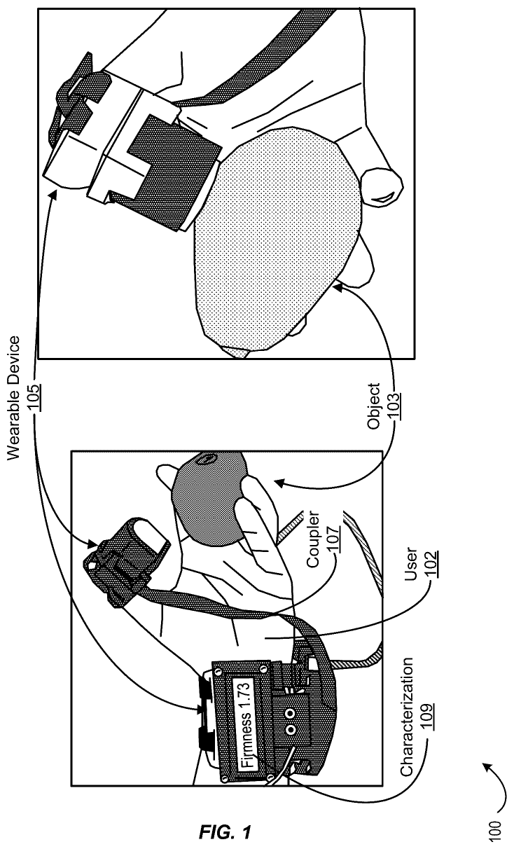

FIG. 1 is a simplified example illustration 100 of a wearable device 105, according to some embodiments. The wearable device 105 is used to determine a firmness (e.g., characterization 109) of an object 103 (e.g., fruit, legume, vegetable, etc.) when an interface of the wearable device 105 contacts the object 103. The wearable device 105 is configured to be worn by a user 102 on a left or right hand. The wearable device 105 may be modular in that two interfaces may be worn on an arm and digit (e.g., thumb) of the user. The two interfaces may be connected by a coupler 107. In some examples, the coupler 107 may include, without limitation, a wired connection, a fabric (e.g., glove, sleave, etc.), a tether (e.g., strap), or combinations thereof. The coupler 107 may be adjustable in length to ensure a good comfortable fit for the user 102 The coupler 107 may be configured to communicate data and relay power to and from the two interfaces.

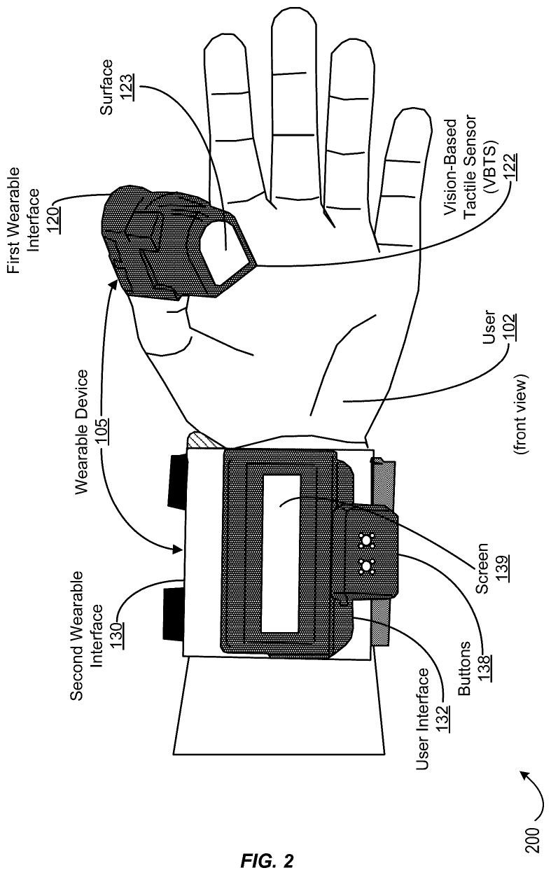

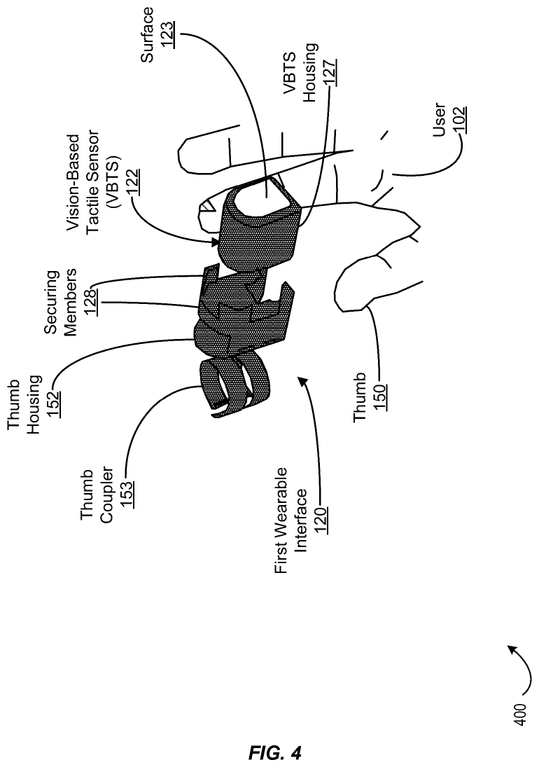

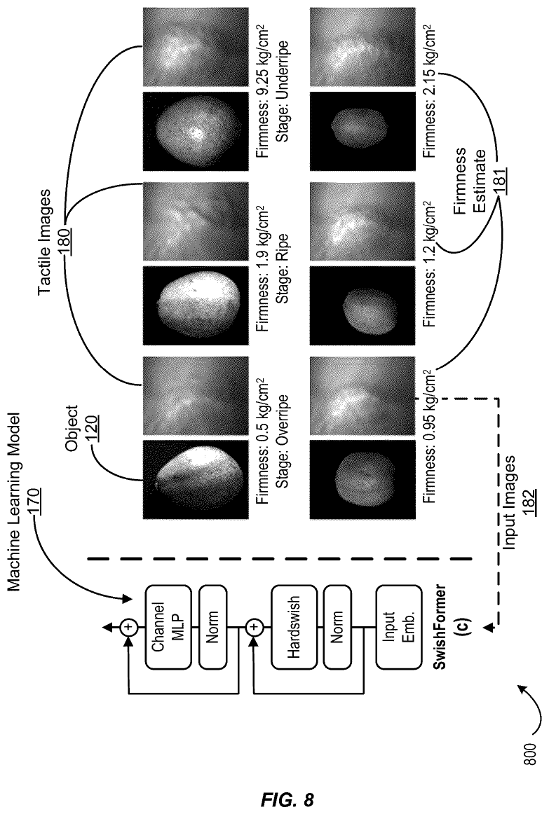

FIG. 2 is a simplified example front view 200 of a wearable device 105, according to some embodiments. The wearable device 105 may include some or all components of the wearable devices 105 with respect to FIGS. 1, 3-7, or 11, or operate according to the methods, processes, or techniques with respect to FIGS. 8-10. The wearable device 105 includes a first wearable interface 120 and a second wearable interface 130 connected together by a coupler (e.g., coupler 107 with respect to FIG. 1). By way of example, the first wearable interface 120 may include a vision-based tactile sensor (VBTS) 122, discussed in more detail with respect to FIG. 4, that includes a surface 123 (e.g., flexible elastomer) that may be configured to physically contact an object (e.g., kiwi, avocado, etc.). The first wearable interface 120 may be worn on a digit (e.g., thumb) of a user 102 (e.g., consumer, quality inspector, etc.) such that the user 102 may grasp the object between the digit and a palm of the user's 102 hand.

The second wearable interface 130 is configured to be worn on an appendage (e.g., a forearm, wrist, etc.) of the user 102. In some examples, the second wearable interface 130 may include a user interface 132 that may include, without limitation, a screen 139 (e.g., a SunFounder™ IC2 liquid crystal display model-1602 (LCD1602) screen) and a user interface 132. The second wearable interface 130 may include one or more buttons 138 (e.g., push buttons, capacitive buttons, switches, actuators, etc.), positioned adjacent or in proximity to the screen 139. The buttons 138 may control one or more parameters of the wearable device 105 including, but not limited to, powering the wearable device 105 into an ON state or an OFF state, controlling when palpation data begins to be collected, controlling when collecting palpation data is to be stopped, screen brightness, data logging, object identification input, taring operations, machine learning training modes (e.g., inputting supervised data), or similar.

In some examples, the first wearable interface 120, including the VBTS, may be worn, or suitably coupled to, any finger of the user's hand 102 or even mounted within a palm of a hand of the user 102. The second wearable interface 130 may be mounted on an upper arm (e.g., bicep), shoulder, any suitable location on a torso, legs, etc. In other examples, the second wearable interface 130 may not be wearable and may be in the form of a client device (e.g., smartphone, laptop, desktop computer, etc.) and/or may be distributed between different locations (e.g., screen 139 on the wrist of a user 102 while the electronics, such as a processor, is located on a server physically distinct from the user 102).

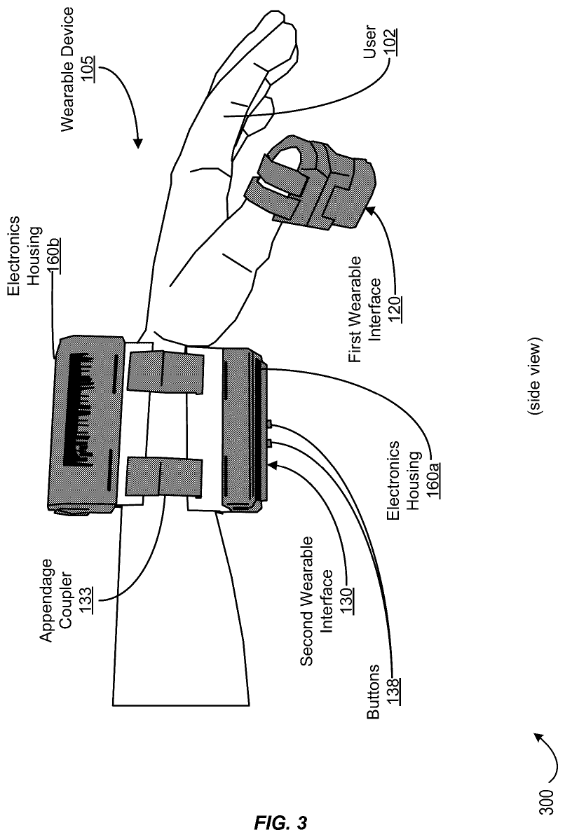

FIG. 3 is a simplified example side view 300 of a wearable device 105, according to some embodiments. The wearable device 105 may include some or all components of the wearable devices 105 with respect to FIGS. 1, 2, 4-7, or 11, or operate according to the methods, processes, or techniques with respect to FIGS. 8-10. The wearable device 105 includes a first wearable interface and a second wearable interface 130 which may include an electronics housing 160a and an electronics housing 160b. In some examples, the electronics housing 160a and the electronics housing 160b may be two or more separate housings or may be integrated as one unit. An appendage coupler 133 may couple the electronics housing 160a and 160b to one another and be configured to receive an appendage of a user 102 therebetween. The appendage coupler 133 may include, without limitation, one or more straps, bands, hook-and-loop fasteners, latches, snap-couplers, press-fit couplers, or combinations thereof. In some examples, the second wearable interface 130 may include one or more buttons 138.

FIG. 4 is a simplified example exploded view 400 of a first wearable interface 120, according to some embodiments. The first wearable interface 120 may include some or all components of the first wearable interface 120 of FIGS. 1-3, 7, or 11, or operate according to the methods, processes, or techniques with respect to FIGS. 8-10. The first wearable interface 120 may include a thumb coupler 153, a thumb housing 152, and a VBTS 122. The thumb coupler 153 may be any suitable coupler such as, but not limited to, straps, bands (e.g., elastic, inelastic, etc.), hook-and-loop fasteners, latches, snap-couplers, press-fit couplers, a digit-glove (e.g., finger fitting glove), a glove, or combinations thereof. The thumb coupler 153 is attached to the thumb housing 152 and is configured to hold the thumb housing 152 in a secure manner on, and/or adjacent, a thumb 150 of a user 102. In some examples, the thumb coupler 153 and the thumb housing 152 are unitary in construction (e.g., cast mold). While the thumb 150 of the user 102 is referenced herein and throughout this disclosure, it should not be considered limiting, and it should be readily recognized by one skilled in the art that any suitable digit (e.g., index, middle, etc.) on a hand of the user 102 may be substituted in lieu of, or in addition to, coupling the first wearable interface 120 on the thumb 150 of the user 102.

The thumb housing 152 may be configured with a shape which substantially matches a contour of an average thumb to provide a secure and comfortable fit. The thumb housing 152 may include securing members 128 (e.g., press-fit couplers, snap-couplers, etc.) configured to couple to or otherwise secure the VBTS 122. The thumb housing 152 may be made of any suitable material such as, but not limited to, metal, alloy, polymers, resin (e.g., 3D-printable resin), composites, or combinations thereof. The thumb housing 152 may have a first end which is adjacent to the thumb 150 of the user 102 and a second end which is adjacent the VBTS 122. In some examples, each of the thumb coupler 153, thumb housing 152, or the VBTS 122 may be modular and readily swapped with suitable equivalents.

As mentioned previously, the VBTS 122 may be received and held securely in place by the thumb housing 152. The VBTS 122 may include a VBTS housing 127 which may house at least one camera (e.g., RGB camera), at least one light source (e.g., light emitted diodes (LED)), a surface 123 (e.g., a flexible elastomer), or electronics (e.g., printed circuit board (PCB)). In some examples, the light source may include, without limitation, at least three colors of light: red, green, blue (RGB). The light source may emit the colors of light in different directions to provide internal illumination for the surface 123 which may be at least partially transparent to at least one wavelength. The camera may be a pixelated camera which may be able to capture one or more color gradients for each pixel for an internally facing side of the surface 123 which is illuminated by the light source. In some examples, the surface 123 may have an outer surface configured to contact the object and an inner surface facing the camera. The outer surface may have a convex shape bowed outward (away from a surface of a thumb) from the VBTS (as depicted with respect to FIG. 1) and an inner surface which may substantially match the contour of the outer surface. The outer surface and the inner surface of the surface 123 may have different coarseness and/or patterns (e.g., protrusions, marks, recesses, ridges, guides, fiducial marks, etc.). The VBTS housing 127 may be substantially opaque to one or more wavelengths of light and provide good confinement of the light source houses therein. In addition, or alternatively, the VBTS 122 may be substantially filled by a transparent elastomer to provide good support to the surface 123. In some examples, the surface 123 may include one or more layers (e.g., absorptive layer, semi-transparent layers, etc.).

In addition, or alternatively, the first wearable interface 120 may include one or more transceivers (not depicted) such as, but not limited to, a wired transceiver (e.g., USB-C, data cable, etc.), a wireless transceiver (e.g., two gigahertz (gHz) to six gHz), a Bluetooth™ transceiver, a ZigBee™ transceiver, cellular, or similar. The first wearable interface 120 may communicate with the second wearable interface 13 wirelessly when a coupler (e.g., coupler 107 with respect to FIG. 1) is not used to serve data/power function. The first wearable interface 120 may include a power source such as a battery, a line-source, or similar.

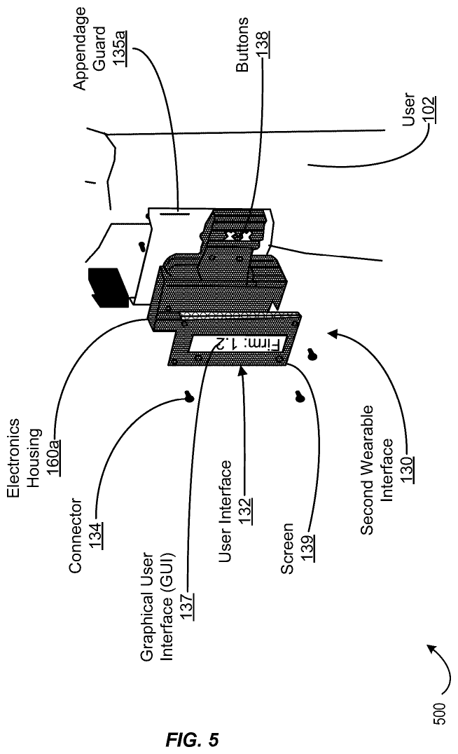

FIG. 5 is a simplified example illustration 500 of a second wearable interface 130, according to some embodiments. The second wearable interface 130 may include some or all components of the second wearable interface 130 of FIGS. 1-3, or 11, or operate according to the methods, processes, or techniques with respect to FIGS. 8-10. The second wearable interface 130 may include an electronics housing 160a coupled to an appendage guard 135a. The electronics housing 160a may be configured to couple to one or more components (e.g., user interface 132, appendage guard 135a, etc.) of the second wearable interface 130, without limitation, by way of connectors 134 (e.g., screws, pins, etc.). The appendage guard 135a may include a first surface which may be contoured to substantially match a shape of a user's 102 appendage such as a forearm or wrist to provide a good and comfortable fit for the user 102 wearing the second wearable interface 130. The electronics housing 160a may be made of any suitable material such as, but not limited to, metal, alloy, polymers, resin (e.g., 3D-printable resin), composites, or combinations thereof. In some examples, the user interface 132 may include a screen 139 configured to display information (e.g., firmness data) on a graphical user interface (GUI) 137 and one or more buttons 138 (as discussed with respect to FIG. 3). The user interface 132 may be modular in that the screen 139, electronics housing 160a, or buttons 138 may be independently swapped or otherwise replaced readily with suitable equivalents. In some embodiments, the user interface 132 may be unitary in construction with the screen 139, electronics housing 160a, or buttons 138. In addition, or alternatively, the user interface 132 may include one or more first transceivers (not depicted) such as, but not limited to, a wired transceiver (e.g., USB-C, data cable, etc.), a wireless transceiver (e.g., two gigahertz (gHz) to six gHz), a Bluetooth™ transceiver, a ZigBee™ transceiver, or similar. The second wearable interface 130 may communicate with the first wearable interface 120 wirelessly when a coupler (e.g., coupler 107 with respect to FIG. 1) is not used to serve a data/power function. The second wearable interface 130 may include a power source such as a battery, a line-source, or similar.

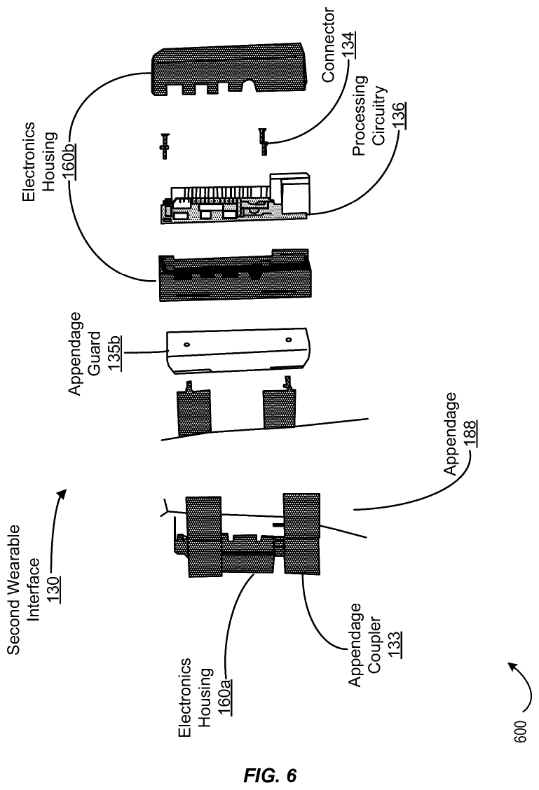

FIG. 6 is a simplified example exploded view 600 of a portion of a second wearable interface 130, according to some embodiments. The second wearable interface 130 may include some or all components of the second wearable interface 130 of FIGS. 1-3, or 11, or operate according to the methods, processes, or techniques with respect to FIGS. 8-10. The second wearable interface 130 may include an electronics housing 160b which may include a first end and a second end. The first end of the electronics housing 160 may be configured to securely couple to an appendage guard 135b. The appendage guard 135b, similar to appendage guard 135a with respect to FIG. 5, may include a complementary contour to a surface of an appendage 188 (e.g., forearm, wrist, etc.) and provide a comfortable secure fit for a user wearing the second wearable interface 130.

In some examples, the appendage guards 135a, 135b may be substantially unitary in construction (e.g., a sleeve) or may be configured to couple the electronics housing 160a to the electronics housing 160b with the appendage 188 therebetween by way of one or more appendage couplers 133. By way of example, the appendage couplers 133 may be any suitable coupler including, but not limited to, one or more elastic or inelastic straps, a fabric coupler, one or more latches, one or more snap-fit couplers, one or more press-fit couplers, or combinations thereof. The appendage couplers 133 may be adjustable in length to ensure a good comfortable fit for the user. The appendage guards 135a, 135b may be made of any suitable material such as, but not limited to, metal, alloy, polymers, resin (e.g., 3D-printable resin), composites, or combinations thereof. In addition, or alternatively, the appendage guards 135a, 135b may include a non-abrasive surface which provides a secure comfortable fit to the user.

Returning to the discussion of the electronics housing 160b, the electronics housing 160b may include one or more electronics components such as, without limitation, processing circuitry 136. The processing circuitry 136 may include one or more memory devices (e.g., RAM, ROM, etc.), one or more processors (e.g., analog, digital, etc.), one or more second transceivers (not depicted) such as, but not limited to, a wired transceiver, a wireless transceiver (e.g., two gigahertz (gHz) to six gHz), a Bluetooth™ transceiver, a ZigBee™ transceiver, or similar. The second transceiver and the first transciever may be configured to communicate with each other. For example, the first transceiver may be configured to capture palpation data and transmit the palpation data to the second transceiver. In addition, the electronics housing 160b may include one or more connectors 134 (e.g., screw, snap-coupler, etc.) for securing the electronics components to the electronics housing 160b.

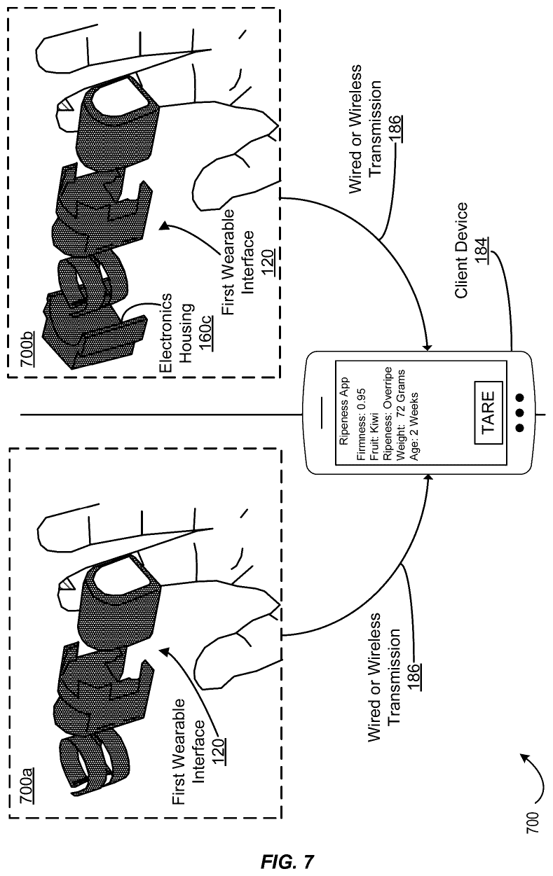

FIG. 7 is a simplified example set 700 of wearable devices 700a and 700b, according to some embodiments. The wearable devices may include some or all components of the first wearable interfaces 120 or the second wearable interfaces 130 of FIGS. 1-7, or 11, or operate according to the methods, processes, or techniques with respect to FIGS. 8-10. By way of example, the wearable device 700a may include one or both of the first wearable interface 120 and the second wearable interface 130 within one or more of the housings for the tactile sensor, thumb housing, or similar, such that the first wearable interface 120 and the second wearable interface 130 are integrated together (not depicted). In this manner, the device profile may smaller, lighter, and more ergonomic. The wearable device 700b may include an electronics housing 160c which may include some or all components (e.g., processing circuitry, one or more PCBs, etc.) of the second wearable device 130. The electronics housing 160c may attach directly to the first wearable interface 120 and provide a good and comfortable fit to a user. One or both of the wearable devices 700a, 700b may include wired or wireless transmission couplers (not depicted) for transmitting and receiving wired or wireless transmissions 186 with a client device 184. The client device 184 may include a smart phone, tablet, or computer, configured with an application (e.g., software) to interface with the wearable devices 700a, 700b for determining fruit firmness. For example, a user may wear the first wearable interface 120 on a digit of their hand (e.g., a thumb) to begin non-destructive palpating motion on a fruit, vegetable, or legume. In this example, the fruit may be still attached to a parent plant (e.g., a tree) to determine firmness. This is called an “on-tree” measurement where the fruit does not need to be removed or otherwise separated from the parent plant to obtain the desired measurements. While this example discusses measuring fruit firmness for a fruit still attached to its parent tree, it is not considered limiting, and the parent plant may be a vine, a bush, a shrub, a root, a branch, or similar.

FIG. 8 is a simplified example group 800 of tactile images 180 used in conjunction with a machine learning model, according to some embodiments. The tactile images 180 may be generated by at least some of the components of the wearable devices 105 with respect to FIGS. 1-7, or 11, and be a result of one or more of the methods, processes, or techniques with respect to FIGS. 8-10. By way of example, a wearable device (e.g., wearable device with respect to FIG. 1) may generate one or more tactile images 180 when a user (e.g., user 102 with respect to FIG. 2) grabs an object 103 (e.g., an avocado) and contacts an outside surface of a VBTS (e.g., surface 123 with respect to FIG. 4) to a surface of the object 103. A camera within the VBTS (as discussed with respect to FIG. 4) may capture an RGB image of an inside surface of the VBTS as the outside surface contacts the object 103. As the object 103 deforms the outside and inside surface of the VBTS, the camera captures the deformation. The tactile images 180 are images of the deformation of the inner surface (and outer surface) of the VBTS caused by contacting the object. Since the inner surface of the VBTS is illuminated by a light source (e.g., LEDs with respect to the discussion of FIG. 4), any deformation of the surface of the VBTS may result in a different tactile image pattern on the inner surface (e.g., a color pattern) due to changing topography.

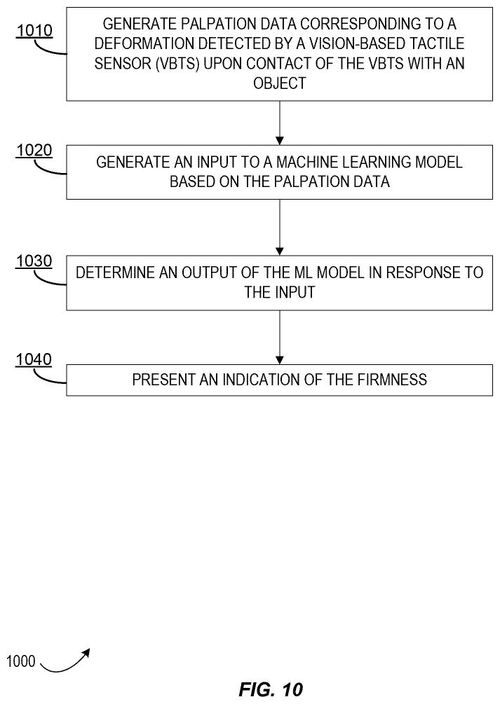

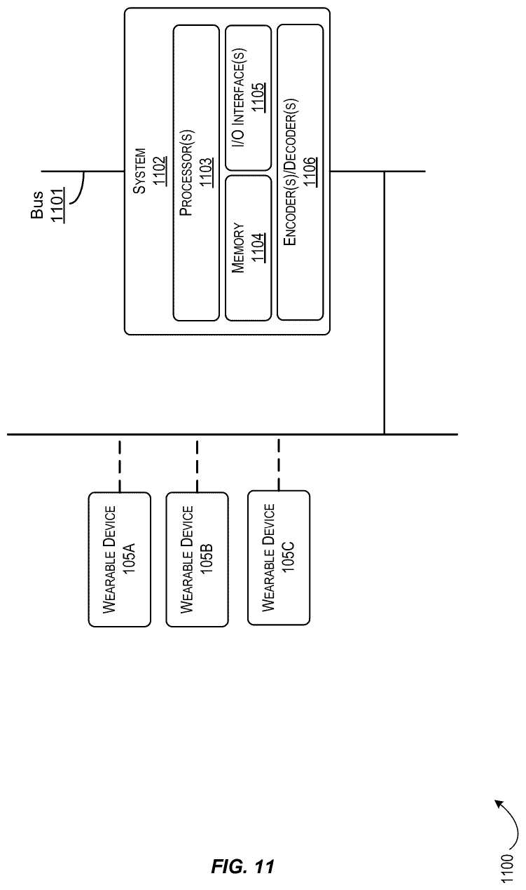

In some examples, one or more calibration images may be captured (e.g., automatically or by user 102 using a user interface 132 with respect to FIG. 5) without the VBTS contacting an object. In this way, the inner surface of the VBTS may be adequately characterized (e.g., identify a rest-state of the inner surface) for comparisons (e.g., correlation, convolution, etc.) with future images captured and/or stored in a memory (e.g., memory 1004 with respect to FIG. 10). In a non-limiting example, once a calibration image (e.g., reference image) has been captured, a user may use the wearable device to hold an object in order for the camera of the VBTS to capture a first set of tactile images 180 for an object (e.g., one or more tactile images). The first set of tactile images may include images from first contact (e.g., the user initially contacting the VBTS to the object) of the outer surface of the VBTS with the object to a time when a palpation signature threshold has been met. The palpation signature threshold may be a temporal threshold where a certain time has been permitted to elapse to capture palpation data or may be a firmness threshold where a controller (e.g., processor(s) 1103 with respect to FIG. 11) determines a firmness estimate 181 is accurate within a percentage (e.g., seventy percent or higher confidence score).

The captured tactile images 180 may be fed as input images 182 into a machine learning model 170 such as SwishFormer (e.g., an unspecified toxen-mixer machine learning model implementing a Hard Swish activation function), convolutional neural networks (CNNs), deep learning models, etc. The machine learning model 170 may be at least partly stored as instructions to be executed by a processor on processing circuitry (e.g., processing circuitry 136) on the wearable device 105 or may be accessed by way of a cloud computing network (e.g., cloud computing network 190 with respect to FIG. 8). For example, when a first tactile image is generated when the VBTS initially contacts the object, at least one tactile image is generated. In addition, or alternatively, for a time interval where the VBTS is continuously, or discretely, contacting the same object, one or more additional tactile images may be generated. The first tactile image and/or additional tactile images may then be fed as input images 182 to the machine learning model, which initiates a token operation (e.g., inputting the input images 182 into the machine learning model as discrete entities) on the input images 182, to generate a firmness estimate associated with the firmness (e.g., how hard or how soft) of the object. In some examples, the input of the machine learning model may be, but not limited to, a single palpation image (e.g., an image with a highest signature), pairs of consecutive images, or a complete video of palpation from the first touch to a last touch to capture deformation of the elasotmer along a temporal distribution. The firmness estimate represents a pressure (e.g., kilograms per centimeter squared (kg/cm²) that the obect exerts on the surface of the VBTS. In some examples, the firmness estimate is calculated, at least in part, us a trained machine learning model which has tactile images stored for various objects at different stages of ripeness.

By way of a non-limiting example, for an avocado, a firmness of 9.25 kg/cm² may indicate an underipe avocado (e.g., an avocado that is not in an optimal state for, without limitation, consumption, shipment, and/or sale). A firmness of 1.9 kg/cm² may indicate that the avocado is ripe and ready for sale and/or consumption. A firmness of 0.5 kg/cm² may indicate that the avocado is overripe (e.g., an avocado that is not in an optimal state for consumption, shipment, and/or sale). Continuing this non-limiting example, but from a perspective of a kiwi, a firmness of 2.15 kg/cm² may indicate an underipe kiwi (e.g., a kiwi that is not in an optimal state for consumption, shipment, and/or sale). A firmness of 1.2 kg/cm² may indicate that the kiwi is ripe and ready for sale and/or consumption. A firmness of 0.95 kg/cm² may indicate that the kiwi is overripe (e.g., e.g., a kiwi that is not in an optimal state for consumption, shipment, and/or sale). Different fruits, vegetables, or legumes may have different firmnesses for determining whether or not the fruit, vegetable, or legume is underripe, ripe, or overripe. The higher the firmness estimate calculated, the “stiffer” the fruits, vegetables, or legumes may be. The lower the firmness estimate calculated, the “softer” the fruits, vegetables, or legumes may be. Typically, the softer the fruits, vegetables, or legumes are, the more ripe they become as they begin to enter a rotting phase. This is not to be considered limiting as one skilled in the art would recognize that while the firmness decreases in the instance of aging fruits, vegetables, and legumes, one skilled in the art would recognize that this downward trend towards softness may not reflect every object to be tested.

The machine learning model 170 performs operations on the input images 182, including, but not limited to, multilayer perceptron (MLP), HardSwish (e.g., piecewise lienar function), or tokenmixing to provide a firmness estimate. In addition, or alternatively, correlation and/or convolutions may be performed on the inputs images 182 to calculate the firmness esimate. For objects that the machine learning model 170 has not been trained on, a user may aid the wearable device in determining ripeness by palpating objects that are known to be underripe, ripe, or overripe to provide a supervised dataset to the machine learning model 170. In addition, or alternatively, the user may add firmness values (e.g., ground truth) for the supervised dataset directly. The user may provide the machine learning model 170 with information such as, but not limited to, a type of object (e.g., kiwi, pineapple, avocado, cucumber, etc.), a ripeness of the object (e.g., ripe, overripe, underripe, rotten, etc.), a lifetime of the object (e.g., how long the fruit has been in a tree or sitting on a shelf), an estimate time of expiration (e.g., how long the user thinks the fruit has until it is not optimal for consumption or sale), or similar. In this way, the machine learning model 170 may learn new objects to aid a user in determining whether the object is in an optimal state. In addition, or alternatively, the machine learning model 170 may receive data from other wearable devices (e.g., wearable devices 105 not being worn by the present user) over a “node-like” network (e.g., a network with respect to FIG. 11) where each wearable device connected over the node-like network may provide each other updated inputs, firmness estimates, look-up tables, or similar.

In addition, or alternatively, the machine learning model 170 may determine a harvest time of the object based on the firmness estimate. For example, avocados grow in trees and are often “picked” from the tree in an underripe stage so that the avocados may continue to ripen while in transit to a commercial facility (e.g., grocery store, restaurant, etc.). A user that uses the wearable device to provide palpation motion to the avocado that the user wants to test may simply grasp (as depicted in FIG. 1) the avocado on the tree without removing the avocado from the tree. The machine learning model 170 will then receive the input images from the first wearable interface (e.g., by way of the VBTS) and begin to calculate the firmness estimate of the avocado. Based on the determined firmness estimate, the wearable device 105 may also reference a look-up table to determine an estimated time predicted to elapse before the object is ripe. In addition, the wearable device may give additional details such as, but not limited to, an estimated expiration time interval of the object, an estimated time interval of ripeness (e.g., how long the fruit may stay ripe), or similar. The estimated intervals may be represented in hours, days, weeks, or months.

According to some embodiments, the machine learning model 170 may identify, based at least partially on the one or more tactile images, a type of object. For example, various objects may impart different deformations on the surface of the VBTS which in turn facilitates the determination of the firmness estimate. Some objects have a larger circumference than others (e.g., a watermelon and a cucumber have wide and narrow contours respectively). The machine learning model 170 may determine, at least in part based on a size of the deformation, a predicted object type. In addition, or alternatively, skin texture information (e.g., obtained by imaging the deformed surface) may be used by the machine learning model 170 to identify fruits, vegetables, or legumes. For example, the machine learning model 170 may be thoroughly trained on identifying ripeness of cucumbers and may recognize repeating patterns (e.g., color patterns) for cucumbers. The machine learning model 170 may also be throughoughly trained on identifying ripeness of avocados and may recognize repeating patterns for avocados and, by way of image correlation, may determine that different objects are being tested. In one such case, consider a customer in a grocery store shopping for fruit. The wearable device of the present disclosure may be available to the customer who may not know if the fruit is ripe. The customer may not be familiar with the intricacies of the wearable device and may simply want to determine ripeness. The customer may grasp an avocado and the wearable device may identify the object type based on the deformation pattern (e.g., any image from the top row of tactile images 180) and then the customer may grasp a kiwi and palpate the kiwi. The machine learning model 170 may determine a firmness estimate as well as identify that a kiwi is being grapsed by the customer wearing the wearable device.

In some examples, the machine learning model 170 may take a type of object into account when determining firmness data (e.g., firmness estimate, expiration time, ripeness time interval). For example, a first apple supplier JohnnyAppleCores may have a first apple type that may have a firmness of 8.25 kg/cm² which may be considered ripe for their proprietary diverse cross-bred apple type with 7.25 kg/cm² being considered overripe for the first apple type, where a time interval from ripe to overripe may be two months. A second apple supplier GrannyJonesApples may have a second apple type that may have a firmness estimate of 8.25 kg/cm² for an underipe apple and a firmness estimate of 7.25 kg/cm² for an overripe apple, where a time interval from underripe to overripe may be three weeks. In this example, if the machine learning model 170 determines a firmness estimate of 8.25 kg/cm², the apple is ripe in the first instance and underripe in the second instance. This may lead to users assuming the fruit is ripe and may lead to early harvesting, early consumption, or similar. To resolve this, the machine learning model 170 may receive, from the user (or by automatic identification), a selection of the type of apple (e.g., JohnnyAppleCores or GrannyJonesApples) and recalculate the firmness data (e.g., ripeness time interval) based on historical data (e.g., by referencing supervised data). The firmness data may the be presented, using the GUI, to the user. For example, the firmness data displayed to the user may include an object identification, a firmness esimate of the object (unchanged), and any suitable information relevant to the object and/or the user such as:
i) “Fruit: JohnnyAppleCore Apple
   Ripeness: Ripe
   Firmness: 8.25 kg/cm²
   Harvest Time: Now
   Ready for consumption.”, or:
ii) “Fruit: GrannyJonesApples Apple
   Ripeness: Underripe
   Firmness: 8.25 kg/cm²
   Harvest Time: One Week
   Not ready for consumption or harvest.”

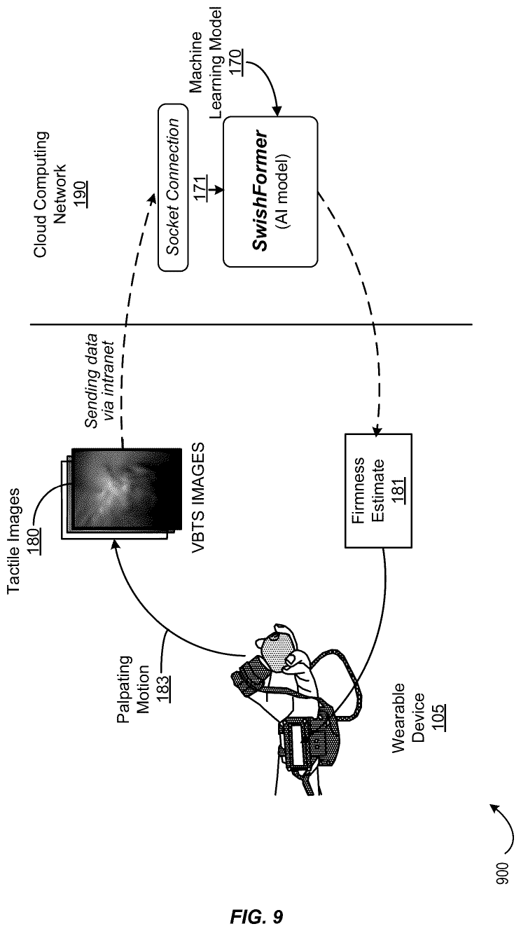

In some examples, the machine learning model 170 may be executed by processing circuitry (e.g., processing circuitry 136 with respect to FIG. 6) coupled to a second wearable interface (e.g., second wearable interface 130 with respect to FIG. 3). In addition, or alternatively, the machine learning model 170 may be executed over a cloud computer network (e.g., cloud computing network 190 with respect to FIG. 9). Outputs from the machine learning model 170, the VBTS, or other wearable devices may be presented on a GUI (e.g., GUI 137 with respect to FIG. 5) on the wearable device. For example, output data related to harvest time of the object, ripeness of the object, firmness estimates of the object, or similar may be presented on the GUI for a user to view and/or interact with. In some examples, comparing the harvest time of the object, determining the ripeness of the object, determining a firmness estimate of the object, or similar, to a threshold (e.g., ripeness threshold, firmness threshold, time threshold) may result in one or more actions being performed. The one or more actions may include, but are not limited to, transmitting a notification (e.g., text message, email, etc.) to a client device (e.g., smartphone, computer, tablet, e tc.), transmitting an notification to one or more wearable devices (e.g., wearable device 105 with respect to FIG. 1), or similar. Then notification may be, without limitation, a visual alert, an audio alert, a haptic alert (e.g., vibration), or similar.

FIG. 9 is a simplified example process 900 for predicting firmness of an object, according to some embodiments. Predicting firmness of an object may be determined by at least some of the components of the wearable devices 105 with respect to FIGS. 1-7, or 11, and be a result of one or more of the methods, processes, or techniques with respect to FIG. 8 or 10. The process 900 begins when a user creates palpating motion 183 by contacting a surface (e.g., surface 123 with respect to FIG. 4) of a VBTS with an object (e.g., a kiwi). The user firmly pressed the surface of the VBTS in order to deform the surface of the VBTS which changes an RGB pattern on an inner surface of the surface of the VBTS which is captured by a camera. These captures (e.g., tactile images 180) are relayed to a second wearable interface (e.g., second wearable interface 130 with respect to FIG. 5) for subsequent transmission over a network (e.g., Internet, intranet, etc.) by a transceiver within the second wearable interface to a cloud computer network 190. The transmission is received by a socket connection 171 of the cloud computing network 190 and then processed by a machine learning model 170 (e.g., as discussed in more detail with respect to FIG. 7). The machine learning model 170 then outputs at least a firmness estimate 181 and subsequently the cloud computer network 190 relays the firmness estimates 191 to the user wearing the wearable device. For example, a user of the wearable device may be in a remote growing field in Florida picking oranges to sell. The user may provide palpation motion to a specific orange under inspection to determine its ripeness. The wearable device may wirelessly relay (e.g., by way of relay stations, cell networks, etc.) the tactile images 180, or data related to thereof, to a computing network (e.g., cloud computing network 190) for the machine learning model 170 to process. Once the machine learning model 170 outputs a result, the network returns the results to the wearable device. In some examples, from beginning palpating motion 183 on the object to receiving and presenting the firmness estimate 181 (e.g., using GUI 137 with respect to FIG. 5) may take twenty seconds or less (e.g., in a range between 0.1 and 20.0 seconds).

FIG. 10 is a simplified example method 1000 for a wearable device, according to some embodiments. The method 1000 may be by at least some of the components of the wearable devices 105 with respect to FIGS. 1-7, or 11, and include steps of one or more of the methods, processes, or techniques with respect to FIG. 8 or 9. The method 1000 may include more or fewer steps than the steps depicted. The steps of the method 1000 may be performed in any suitable order.

The method may begin at step 1010 where palpation data corresponding to a deformation maybe detected by a vision-based tactile sensor (VBTS) upon contact of the VBTS with an object (e.g., a kiwi, object 103 with respect to FIG. 1). For example, a user wearing a wearable device (e.g., wearable device 105 with respect to FIG. 3) may grasp an object in their hand (as depicted in FIG. 1) and palpate the object with the VBTS coupled to the users thumb. The object may be a fruit, legume or vegetable. The VBTS includes a surface which deforms when the object contacts it.

The method may continue at step 1020 where an input may be generated to a machine learning model based on the palpation data. In some examples, the machine learning model is executed locally on the wearable device. In addition, or alternatively, the machine learning model may be executed at least partially on a system remote from the wearable device. For example, the palpation data (input) generated by the VBTS may be sent to the system (e.g. cloud computing network 190 with respect to FIG. 9) for processing.

The method may continue at step 1030, where the machine learning model may generate an output in response to the palpation data. For example, the wearable device may then receive the output from the machine learning model (e.g., machine learning model 170 with respect to FIG. 8) in order to present the firmness estimate to the user. The output may be generated on the wearable device or may be received from a system remote from the wearable device.

The method may continue at step 1040, where an indication of the firmness of the object may be presented to the user. For example, the indication may include a firmness estimate (e.g., firmness estimate 181) of the object. In addition, or alternatively, a characterization of the firmness estimate may be presented to the user. For example, the characterization may include, without limitation, text such as “Ripe”, “Underripe”, or “Overripe”.

FIG. 11 is a simplified example network interface 1100 for a wearable device, according to some embodiments. One or more of the wearable devices 105A-105C may be the same and include some or all components and structural dimensions as wearable devices 105 of FIGS. 1-7 and operate according to some or all methods, processes, or techniques with respect to FIGS. 8-10. The wearable devices 105A-105C may be coupled to one or more system 1102. By way of example, one of the wearable devices 105A-105C may generate input palpation data to be relayed to the system 1102 (e.g., wired, wirelessly, etc.). The wearable devices 105A-105C may perform one or more operation(s) on the input palpation data (e.g., as in the operations in FIG. 8-10) and relay the data to system 1102. In some examples, the wearable devices 105A-105C may include any suitable number of wearable devices 105A-105C connected in a “node-like”network.

In some examples, the wearable devices 105A-105C and components of system 1102 may be coupled to a communication bus 1101 (e.g., wired connection, wireless connection, etc.) to transmit signals. The components may include one or more processor(s) 1103, non-transitory computer readable medium(s) such as memory 1104, an input/output (I/O) interface(s) 1105, and/or encoder(s)/decoder(s) 1106. The one or more processor(s) 1103 may execute machine-readable instructions stored on the memory 1104. The one or more processor(s) 1103 may include single core or multi-core processors, Raspberry Pi 4, etc. The memory 1104 may be configured in any suitable configuration. For example, memory 1104 may be volitile memory such as random access memory (RAM) and/or non-volatile memory such as read-only memory (ROM) and/or flash memory. In addition, or alternatively, the one or more processor(s) 1103 and/or memory 1104 may function with the I/O interface(s) 1105 to receive signals from the wearable devices 105A-105C. The I/O interface(s) 1105 may include any suitable interface including user interfaces such as computers, controllers (e.g., keyboard, mouse, etc.), or similar. In some examples, encoder(s)/decoder(s) 1106 may function to receive signals from the wearable devices 105A-105C. The encoder(s)/decoder(s) 1106 may encode the signals for further communication or may decode the signals for analysis and/or storing in memory 1104 and provide secure communications for wearable devices 105A-105C.

As used in this application and in the claims, some or all devices, methods, and apparatus discussed herein may be components in one or more networks for connecting communication paths. For example, the wearable devices 105A-105C discussed herein may be used for receiving and/or transmitting data packets to and/or from one network to another network. Multiple wearable devices 105A-105C may be implemented with the one or more networks and work in conjunction with each other. The networks may include software, hardware, or firmware to operate with the wearable devices 105A-105C. In some examples, networks may include, but are not limited to, wide area networks (WAN) (e.g., the Internet), local area networks (LAN) (e.g., university networks), virtual private networks (VPN), internet of things (IoT) networks, any appropriate network/cloud architecture that may facilitate data communications, or combinations thereof.

As used in this herein and in the claims, the terms first, second, etc., are intended to distinguish the particular nouns they modify (e.g., first image, second image.) and should not be considered limiting. The use of these terms is not intended to indicate any type of importance, hierarchy, preference of the particular noun. For example, a first image and a second image are intended to demonstrate two separate images that are not necessarily limited by any importance, hierarchy, preference of the two images.

As used in this application and in the claims, the singular forms “a”, “an”, and “the” include the plural forms unless the context clearly dictates otherwise. Additionally, the term “includes” means “comprises”. Further, the terms “couple” or “coupled” or “support” or “supported” does not exclude the presence of intermediate elements between the coupled items and/or supported items.

The devices, methods, systems, processes, and/or techniques described herein should not be considered limiting in any way. Instead, the present disclosure is directed toward all non-obvious and novel features and aspects of the various disclosed embodiments, alone and in various combinations and sub-combinations with one another. The disclosed devices, methods, systems, processes, and/or techniques are not limited to any specific aspect or feature or combinations thereof, nor do the disclosed devices, methods, systems, processes, and/or techniques require that any one or more specific advantages be present. Any theories of operation are to facilitate clear and direct explanation, but the disclosed devices, methods, systems, processes, and/or techniques are not limited to such theories of operation.

Although the operations of some of the disclosed methods are described in a particular, sequential order for convenient presentation, it should be understood that this manner of description encompasses any suitable rearrangement, unless a particular ordering is preferred and/or required by specific language set forth below. For example, operations described sequentially may in some cases be rearranged or performed concurrently. Moreover, for the sake of simplicity, the attached figures may not show the various ways in which the disclosed systems, methods, and apparatuses can be used in conjunction with other devices, methods, systems, processes, and/or techniques. Additionally, the description sometimes uses terms like “produce” and “provide”, and similar to describe the disclosed methods. These terms should be considered as high-level abstractions of the actual operations that are performed. The actual operations that correspond to these terms will vary depending on the particular implementation and are readily discernible by one of ordinary skill in the art. Moreover, the description sometimes uses terms like “substantially”, “approximately”, and similar to describe the disclosed devices and apparatus. These terms may represent an equivalence readily understood to one skilled in the art to within a specific percentage (e.g., +/−five percent, +/−ten percent, etc.) for comparison of structures, ratios, dimensions, ranges, operations, or similar.

In some examples, structural elements, geometric relationships, thresholds, criteria, values, procedures, or apparatuses are referred to as “low”, “minimal”, “optimal”, or the like. It will be appreciated that such descriptions are intended to indicate that a selection among many used functional alternatives can be made, and such selections need not be better, smaller, or otherwise preferable to other selections.

What is claimed is:

1. A method comprising:
generating, by using a vision-based tactile sensor (VBTS) of a wearable device, palpation data corresponding to a deformation detected by the VBTS upon a contact of the VBTS with an object that includes at least one of: a fruit, a legume, or a vegetable;
generating an input to a machine learning (ML) model based on the palpation data;
determining an output of the ML model in response to the input, wherein the output indicates a firmness of the object; and
presenting, by using a user interface (UI) of the wearable device, an indication of the firmness.

2. The method of claim 1, wherein the input includes the palpation data, and wherein the method further comprises:
displaying, by the UI, a characterization of the firmness of the fruit, legume, or vegetable.

3. The method of claim 1, wherein the palpation data is generated based on the VBTS being in contact with the object without the object being removed from a parent plant to which object is attached and without a damage to the object.

4. The method of claim 1, wherein the ML model is executed on a system remote from the wearable device, wherein the method further includes:
sending the palpation data as the input to the system; and
receiving the output of the ML model from the system.

5. The method of claim 1, further comprising:
executing, locally on the wearable device, the ML model.

6. The method of claim 1, wherein a time interval between generating the palpation data and presenting the indication is in a range between 0.1 and 20.0 seconds.

7. A device comprising:
a first wearable interface;
a vision-based tactile sensor (VBTS) attached to the first wearable interface and configured to generate palpation data upon contact of the VBTS with an object that includes at least one of a fruit, legume, or vegetable;
a second wearable interface; and
a user interface (UI) attached to the second wearable interface and configured to present an indication of a firmness of the object, the indication generated by a machine learning model (ML) based on the palpation data.

8. The device of claim 7, further comprising:
processing circuitry attached to the second wearable interface and configured to send the palpation data to a system executing the ML model, wherein the processing circuitry is configured to receive the firmness as an output of the ML model and cause the presentation of the firmness at the UI.

9. The device of claim 7, further comprising:
processing circuitry attached to the second wearable interface and configured to execute the ML model.

10. The device of claim 7, wherein the first wearable interface further comprises:
a housing configured to receive a thumb at a first end, wherein the housing is configured to receive the VBTS at a second end; and
at least one camera.

11. The device of claim 7, wherein the second wearable interface further comprises:
a housing configured to couple to an appendage;
a screen configured to display a graphical user interface (GUI) and at least partially disposed within the second wearable interface, wherein the UI includes the GUI; and
wherein the second wearable interface is configured to at least partially house processing circuitry and a transceiver.

12. The device of claim 7, wherein the first wearable interface and the second wearable interface are linked together by a coupler selected from a group comprising: a wired connection, a wireless connection, a fabric, a tether, or combinations thereof.

13. The device of claim 7, wherein the VBTS includes a surface configured to contact the object, and wherein the surface is an elastomer configured to deform when placed in contact with the object.

14. A non-transitory computer readable medium having stored thereon computer-readable instructions that, when executed by a processor, cause the processor to perform operations comprising:
generating, by using a vision-based tactile sensor (VBTS), palpation data corresponding to a deformation detected by the VBTS upon a contact of the VBTS with an object;
generating an input to a machine learning (ML) model based on the palpation data;
determining an output of the ML model in response to the input, wherein the output indicates a firmness of the object; and
causing a presentation of an indication of the firmness at a user interface (UI).

15. The non-transitory computer readable medium of claim 14, wherein the object is a fruit or vegetable; and wherein the operations further comprise:
determining a harvest time of the object based on the firmness, wherein the harvest time is an estimated time predicted to elapse before the object is ripe; and
presenting, using the UI, the harvest time.

16. The non-transitory computer readable medium of claim 15, wherein the operations further comprise:
storing the harvest time in a memory; and
in response to comparing the harvest time to a time threshold, transmitting a notification to a client device.

17. The non-transitory computer readable medium of claim 14, wherein the palpation data includes one or more tactile images; and wherein the operations further comprise:
receiving, from the VBTS, a first tactile image when VBTS initially contacts the object;
receiving, from the VBTS, additional tactile images while VBTS contacts the object during a time interval, wherein the one or more tactile images includes the first tactile image and the additional tactile images; and
causing a token operation to be performed by the ML model on the one or more tactile images to generate firmness data associated with the firmness of the object.

18. The non-transitory computer readable medium of claim 17, wherein the operations further comprise:
identifying, based at least partially on the one or more tactile images, a type of the object;
in response to identifying the type, recalculating the firmness data; and
presenting, using the UI, the type and the recalculated firmness data.

19. The non-transitory computer readable medium of claim 17, wherein the operations further comprise:
causing training of the ML model using the one or more tactile images; and
in response to the training, updating the firmness data.

20. The non-transitory computer readable medium of claim 14, wherein the operations further comprise:
receiving, from the UI on a wearable device, a selection of a type of the object;
in response to receiving the type of the object, recalculating the firmness; and
presenting, using the UI, the recalculated firmness.
